(19) United States
(12) Patent Application Publication
(10) Pub. No.: US 2026/0020967 A1
(43) Pub. Date: Jan. 22, 2026

(54) PROCESS FOR PRODUCING AN ANATOMICAL PROSTHESIS COMPUTER-AIDED THREE-DIMENSIONAL DESIGN

(71) Applicant: ATELIER J'M SASU, Paris (FR)

(72) Inventor: Julien MONTENERO, Levallois-Perret (FR)

(73) Assignee: ATELIER J'M SASU, Paris (FR)

(21) Appl. No.: 18/850,524

(22) PCT Filed: Mar. 24, 2023
(86) PCT No.: PCT/EP2023/057745

§ 371 Date: Sep. 24, 2024

(30) Foreign Application Priority Data
Mar. 24, 2022 (FR) FR2202659

(51) Int. Cl. A61F 2/52; A61F 2/50

(52) U.S. Cl. CPC A61F 2/52; A61F 2/5046; A61F 2002/505

(57) ABSTRACT

The invention relates to a method of producing an anatomical prosthesis made of flexible synthetic material by computer-aided three-dimensional design on the basis of a first digital volumetric scanscan obtained by three-dimensional digitisation performed by three-dimensional surface scanning of an envelope of the volume of a part of the body of a patient before ablation of an anatomical part to be replaced by the anatomical prosthesis, and of a second digital volumetric scan obtained by three-dimensional digitisation carried out by the same technique of the same part of the body of the patient after ablationablation of the anatomical part to be replaced by the anatomical prosthesisablation. In the method for producing an anatomical prosthesis, a digital volumetric representation of an envelope of the volume of the anatomical prosthesis is obtained from the superposition of the first and second digital volumetric scans in order to reproduce identically an envelope of the anatomical part to be replaced by said anatomical prosthesis.

### TECHNICAL SCOPE OF THE INVENTION

The invention generally concerns the technical field of anatomical prostheses designed to repair, restore or reinstate anatomical parts for patients who have lost a limb or part of their body in order to restore them to a so-called normal human appearance.

It also concerns processes for producing anatomical prostheses made of flexible or composite synthetic material with flexible and rigid parts.

The invention applies, in particular, to epitheses, facial prostheses, maxillofacial prostheses, and external breast prostheses to be inserted into a pocket of an underwear or to be placed directly, for example, by medical adhesive or by self-adhesive sheets, on the skin after complete healing following a total or partial mastectomy.

### STATE OF THE ART

Prostheses made of synthetic materials are known and are generally delivered after mutilation or ablation by surgical act following accidents or in invasive cancer pathologies requiring a wide or limited exegesis. In these conditions, patients need a prosthesis to replace the anatomical part that has been removed in order to regain their physical integrity and an appearance that is as identical as possible to their previous appearance, and to be able to continue a normal social life.

The process consists of replacing anatomical parts removed as a result of any circumstance requiring the prescription of an anatomical prosthesis with a made-to-measure prosthesis, usually made of silicone, for example following cancer, trauma, burns, congenital diseases, or mutilation as a result of assault or war.

Every year, 20,000 women in France and 720,000 worldwide undergo major surgical procedures with irreversible consequences following the diagnosis of invasive breast cancer. In most cases, these cancerous pathologies require one or both breasts to be removed to a greater or lesser extent, known as a mastectomy. This is known as a bilateral mastectomy. In some cases, ablation is limited to a specific area where a tumour has developed. This area is most often located behind the nipple. This limited surgical procedure is called lumpectomy. The psychological consequences of this type of mutilation have a severe impact on patients, who may turn to various techniques to regain their pre-surgery appearance and resume a normal social life.

Sometimes reconstructive surgery will be performed, but in the case of a partial mastectomy or lumpectomy, for example, the original volume of the breast may be restored, but not the nipple or areola, which are important parts of a breast's appearance. Some patients refuse reconstruction or it is not medically possible.

There are standard prostheses with different volumes of predetermined sizes and different colours in a standard range. Because these prostheses are standard, they will never be exactly identical to the patient's morphology prior to surgery. Furthermore, these standard prostheses are exclusively intended to be inserted into a pocket of an underwear adapted for this purpose.

In order for the greatest number of patients who have undergone mutilation or surgical ablation, such as a mastectomy, to be able to rebuild psychologically and physically following this trauma, there is a need for a breast prosthesis, or other prosthesis, which identically reproduces the anatomical part removed to replace it, but which is affordable for the majority of patients. To this end, a process for producing anatomical prostheses identical to the anatomical part removed is needed to replace existing standard prostheses.

Patent application DE102018006839 A1 discloses a method for producing digital scans of the part of the body to be ablated before and after surgery. These pre- and post-operative digital scans are obtained from DICOM files produced by the usual medical imaging techniques, such as mammography, sonography, tomography, nuclear magnetic resonance, medical scanner, etc. These DICOM files form part of the patient's medical file and have been produced by medical teams before and after the surgical procedure. The DICOM files are then transcribed into STL files so that they can be used by a 3D printer. The accuracy of the files obtained using medical imaging techniques is not sufficient for a good visual rendering of the prosthesis.

The aim of the present invention is to remedy all or some of the disadvantages of the state of the art by proposing, in particular, to use and exploit the current technologies of three-dimensional digitisation, computer-assisted 3D design and 3D printing to form a chain of digital technologies for producing prostheses personalised to each patient, in order to reproduce his or her original morphology.

To this end, according to a first aspect of the invention, a method is proposed for producing an anatomical prosthesis made of flexible synthetic material by computer-assisted three-dimensional design on the basis of a first digital volume record obtained by three-dimensional digitisation performed by three-dimensional surface scanning of an envelope of the volume of at least one part of a patient's body before ablation of an anatomical part to be replaced by the said anatomical prosthesis, or by digital simulation of the envelope of the volume of at least one part of the body of a patient before ablation of an anatomical part to be replaced by the said anatomical prosthesis, and of a second digital volume record obtained by three-dimensional digitisation carried out by three-dimensional surface scanning of an envelope of the volume of the same said at least one part of the body of the patient after ablation of the anatomical part to be replaced by the said anatomical prosthesis. In this method, a digital volumetric representation of an envelope of the volume of the anatomical prosthesis is obtained from the superposition of the first and second digital volumetric scans so as to reproduce identically an envelope of the anatomical part to be replaced by said anatomical prosthesis.

Preferably, the external visual appearance of the anatomical prosthesis reproduces identically the visual appearance of the anatomical part to be replaced by said anatomical prosthesis on the basis of a digital colorimetric scan and/or a centimetric photographic scan of the anatomical part to be replaced by said anatomical prosthesis before its ablation, preferably said digital colorimetric scan and/or said centimetric photographic scan completes said digital volumetric representation; and/or the density of the volume of the said anatomical prosthesis is defined on the basis of a digital tomographic scan of at least one part of the patient's body taken before ablation of the anatomical part to be replaced by the said anatomical prosthesis, preferably the said digital colorimetric scan and/or the said centimetric photographic scan completes the said digital volume representation.

Advantageously, a base of the said anatomical prosthesis oriented towards the patient's skin is fused to a pattern whose size corresponds to that of the said base of the anatomical prosthesis; or a base of the said anatomical prosthesis intended to be in contact with the patient's skin has a topography obtained from the said second digital volume scan so as to correspond identically to the profile of an area of the patient's body from which the anatomical part to be replaced by the said anatomical prosthesis has been excised.

Preferably, a mould is made on the basis of said digital volume representation, into which the flexible synthetic material is injected to form said volume of said anatomical prosthesis.

Advantageously, the mould is produced by additive manufacturing from the digital volume representation.

Preferably, the flexible synthetic material injected into the mould has a colour corresponding to the digital colorimetric scan, and/or a density corresponding to the average density of the digital tomographic scan, or a density distribution derived from the digital tomographic scan.

Optionally, after moulding the anatomical prosthesis, a finishing step reproduces the pigmentation of the different areas of the volume of said anatomical prosthesis identical to the anatomical part to be replaced by the anatomical prosthesis on the basis of the digital and/or centimetric photographic colour measurement.

Alternatively, the anatomical prosthesis is produced by additive manufacturing on the basis of the said digital volume representation so that the shape of the volume of the said anatomical prosthesis is identical to the shape of the anatomical part to be replaced by the said anatomical prosthesis, the said digital colourimetric measurement and/or the said digital photographic measurement so that the visual appearance of the said anatomical prosthesis is identical to the visual appearance of the anatomical part removed, and/or the digital tomographic measurement so that the density distribution in the said anatomical prosthesis is identical to the density distribution in the anatomical part to be replaced by the said digital prosthesis.

Advantageously, the flexible synthetic material used is a biomedical silicone dyed in the mass in order to reproduce the colours with the tone and saturation level required by the digital colour measurement.

According to a second aspect of the invention, an anatomical prosthesis produced by moulding or by additive manufacturing is proposed, characterised in that it is produced on the basis of a digital volume representation of an anatomical prosthesis volume obtained by superimposing a first digital volume record obtained by three-dimensional digitisation produced by three-dimensional surface scanning of an envelope of the volume of at least one part of a patient's body before ablation of an anatomical part to be replaced by the said anatomical prosthesis, or by digital simulation of the envelope of the said at least one part of a patient's body before ablation of an anatomical part to be replaced by the said anatomical prosthesis, and of a second digital volume record obtained by three-dimensional digitisation produced by three-dimensional surface scanning of an envelope of the volume of the same said at least one part of the patient's body after ablation of the anatomical part to be replaced by the said anatomical prosthesis.

Preferably, the external visual appearance of the anatomical prosthesis reproduces identically the visual appearance of the anatomical part to be replaced by the said anatomical prosthesis on the basis of a digital colorimetric scan and/or a centimetric photographic scan of the anatomical part to be replaced by the said anatomical prosthesis; and/or the density of the volume of the said anatomical prosthesis is defined on the basis of a digital tomographic scan of at least one part of the patient's body carried out before ablation of the anatomical part to be replaced by the said anatomical prosthesis.

According to a third aspect of the invention a method is proposed for modelling an anatomical prosthesis by computer-aided three-dimensional design on the basis of a digital modelling file comprising first data for digital volume representation of an envelope of the anatomical prosthesis obtained by superimposing a first digital volume scan obtained by three-dimensional digitisation performed by three-dimensional surface scanning of an envelope of the volume of at least one part of a patient's body before ablation of an anatomical part to be replaced by the said anatomical prosthesis, or by digital simulation of the envelope of the volume of at least one part of a patient's body before ablation of an anatomical part to be replaced by the said anatomical prosthesis, or by digital simulation of the envelope of the volume of at least one part of a patient's body before ablation of an anatomical part to be replaced by the said anatomical prosthesis, or by digital simulation of the envelope of the volume of at least one part of the body of a patient before ablation of an anatomical part to be replaced by the said anatomical prosthesis, and of a second digital volume record obtained by three-dimensional digitisation produced by three-dimensional surface scanning of an envelope of the volume of the same said at least one part of the body of the patient after ablation of the anatomical part to be replaced by the said anatomical prosthesis.

Advantageously, the digital modelling file also includes second data corresponding to a digital colorimetric scan and/or a centrimetric photographic scan of the anatomical part to be replaced by the said anatomical prosthesis before its ablation, and/or third data corresponding to a digital tomographic scan of at least one part of the patient's body carried out before ablation of the anatomical part to be replaced by the said prosthesis.

Other features and advantages of the invention are highlighted by the following description of non-limiting examples of the various aspects of the invention.

### BRIEF DESCRIPTION OF FIGURES

The description refers to the attached figures, which are also given as non-limiting examples of the invention:

FIG. 1a shows a representation of part of a patient's body before mastectomy;

FIG. 1b shows a representation of part of a patient's body after mastectomy;

FIG. 1c shows a representation of a volume scan before mastectomy;

FIG. 2a illustrates the device for superimposing volume scans before and after mastectomy;

FIG. 2b illustrates the tomographic scan of the breast to be removed;

FIG. 3 illustrates a first production process for the anatomical prosthesis;

FIG. 4 shows a cross-sectional view of a breast prosthesis in a first embodiment;

FIG. 5 illustrates a second process for producing the anatomical prosthesis;

FIG. 6 shows a cross-sectional view of a breast prosthesis in a second embodiment;

FIG. 7 shows a provisional breast prosthesis;

FIG. 8 shows a nipple and areolar prosthesis; and

FIG. 9 shows a covering breast prosthesis.

For greater clarity, identical or similar elements are identified by identical reference signs throughout the figures.

### DETAILED DESCRIPTION OF A PRODUCTION METHOD

In the remainder of this description, the invention will be described mainly with reference to breast prostheses delivered after total or partial mastectomy of one or both breasts. Nevertheless, the principles and techniques implemented and described below apply mutatis mutandis to any type of prosthesis in the situations indicated above. Thus, the invention can also be applied to limb replacement prostheses after amputation, epitheses, facial prostheses and maxillofacial prostheses.

The solution proposed by the invention consists of representing and reproducing identically the anatomical part that has been removed, for example a breast after mastectomy, in order to replace it. In addition to the technical nature of the procedures involved in manufacturing such an anatomical prosthesis, certain stages are also designed to listen to and understand the trauma suffered by patients.

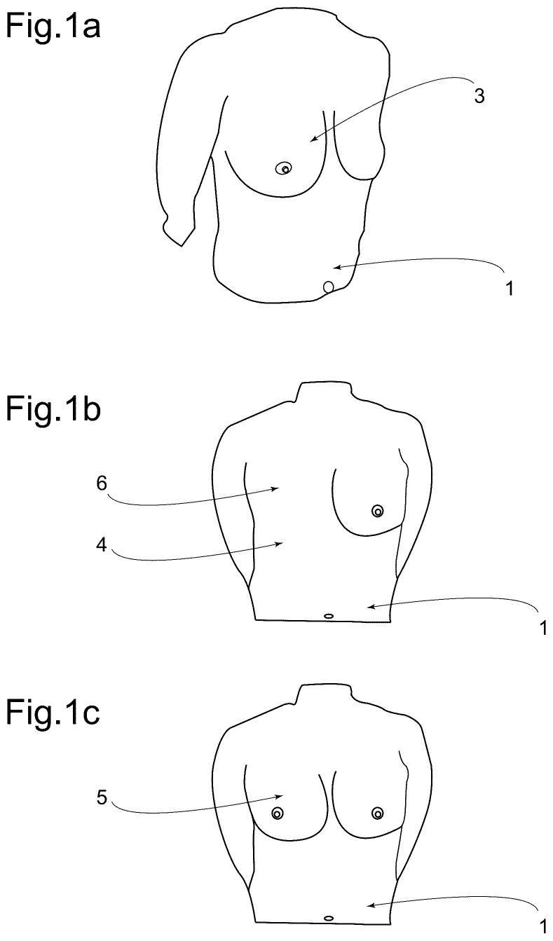

As shown in FIG. 1a [FIG. 1a], the process for producing the anatomical prosthesis begins with an initial three-dimensional surface scan of the area of the patient's body in which the anatomical part to be removed, in this case a breast, is located. This initial three-dimensional surface scan takes place during an initial appointment with the patient at the prosthetist's office, during which various prosthesis proposals are presented to her using computer-generated images produced on the basis of the initial three-dimensional surface scan, for example simulations of temporary and permanent prostheses. This three-dimensional surface scan is carried out using a 3D surface scanner or any other three-dimensional volume digitisation technique that transcribes the scanned anatomical volumes into a digital file. After the surgical operation, in the example of implementation of the invention described below a mastectomy, a new operation is performed to carry out a second three-dimensional surface scan.

The three-dimensional surface scanner or 3D scanner is used to produce 3D scans using a technique that captures the shape of an object or anatomical part. The result is a 3D computer file that can be saved, edited and even printed in 3D. There are several technologies available for 3D scanning of objects, environments or people. The 3D scanner analyses an object and produces a cloud of points on the surface of the object to be scanned in order to digitise its outer envelope, i.e. its volume. This 3D scan is processed by 3D software to obtain an accurate reproduction of the scanned object. Most 3D scanners use triangulation laser technology or structured light.

The data resulting from a 3D scan, i.e. the coordinates of the measured points belonging to the envelope of the object, are stored in a file in STL format. OBJ, PLY, etc. Non-contact 3D scanners represent a non-invasive technical solution for accurately recording anatomical parts of a body without altering the tissue, in order to capture in digital form the exact shape of the volume envelope of the anatomical part scanned. Furthermore, these digital files can be easily transposed to produce a 3D print of the scanned object or anatomical part.

Examples of 3D surface scanners that can be used for the applications described below are strutted light scanners such as those from ARTEC (ARTEC EVA-LEO and SPIDER). Smartphone or tablet scanners can also be used. They all record the volume envelope of the anatomical part scanned and produce an STL, OBJ, PLY file, etc. These digital files model an anatomical volume as required and then allow various digital processes to be carried out, such as enrichment with additional data or 3D printing. At the first appointment with the patient before the mastectomy, the skin colours of the breast to be replaced by the anatomical prosthesis are recorded in addition to the initial three-dimensional record. This skin colour scan is carried out using, for example, a spectrophotometer, such as a Spectroshade®, or a colorimeter, such as a Datacolor Coloreader EZ®. These colour measurements are used to record the skin tone of the anatomical part to be removed so that the anatomical prosthesis can reproduce it faithfully. At the same time, centimetric photographic measurements are also taken to record any details visible on the surface of the breast to be removed, such as freckles, veins, etc.

As illustrated in FIG. 1a [FIG. 1a], whenever possible, an initial digital volume scan of an upper part of the body of patient 1 is carried out, optionally with the other colorimetric and photographic measurements indicated above. The aim of this first digital volume scan is to obtain a three-dimensional image of the volume envelope of the anatomical parts by 3D surface scanner in order to reproduce the anatomical volumes identically before any transformation of this part of the body of patient 1 following a mastectomy.

The first procedure therefore involves making an initial 3D digital impression of at least one part of the patient's body in order to record all the anatomical details that existed before the mastectomy. This first 3D digital impression is used to obtain the first digital volume record 3. In the case of a breast mastectomy, the digital volume record represents the digital transcription of the envelope of the exact volumetric part of the breast with an accuracy of approximately 100 microns. In this way, the envelope of the exact volumes of the patient's areola and nipple will be recorded in order to methodically transcribe the real volumes that an anatomical prosthesis, in this case a breast prosthesis, must reproduce.

FIG. 1b [FIG. 1b] illustrates a second phase of the procedure which takes place after the mastectomy. During a second appointment with the patient after the mastectomy at the prosthetist's office, a second three-dimensional surface scan of the same upper part of the body of patient 1 after ablation of the breast is carried out. This second three-dimensional surface scan is carried out using the same technique as the first and makes it possible to produce a second 3D digital impression of the upper part of the body of patient 1 after mastectomy in the form of a second digital volume scan 4. The aim of this second three-dimensional surface scan is to obtain a three-dimensional scan of the envelope of the body volume when the ablation has taken place. In particular, the second digital volume scan 4 makes it possible to record the topography of the part of the patient's body that has undergone the mastectomy, and which contains a scar 6 following the mastectomy.

Then, as shown in FIG. 1c [FIG. 1c], the first and second digital volume measurements 3 and 4 are superimposed by digital “matching” of the two files. This produces a transparent digital volume representation 5 of the volume envelope of the removed anatomical part that is to be replaced by the anatomical prosthesis. In this way, the anatomical prosthesis to be designed can be obtained using three-dimensional computer-aided design software that formalises the volume envelope of the anatomical part that is missing as a result of the surgical procedure. This three-dimensional CAD software uses the files of the first and second digital volume scan 3 and 4 to transpose the data contained in these files representing the scanned volumes to obtain the digital volume representation 5 illustrated in FIG. 1c [FIG. 1c] on the basis of which the volume envelope of a 3D anatomical part can be drawn and its volumes reproduced by 3D printer as described below.

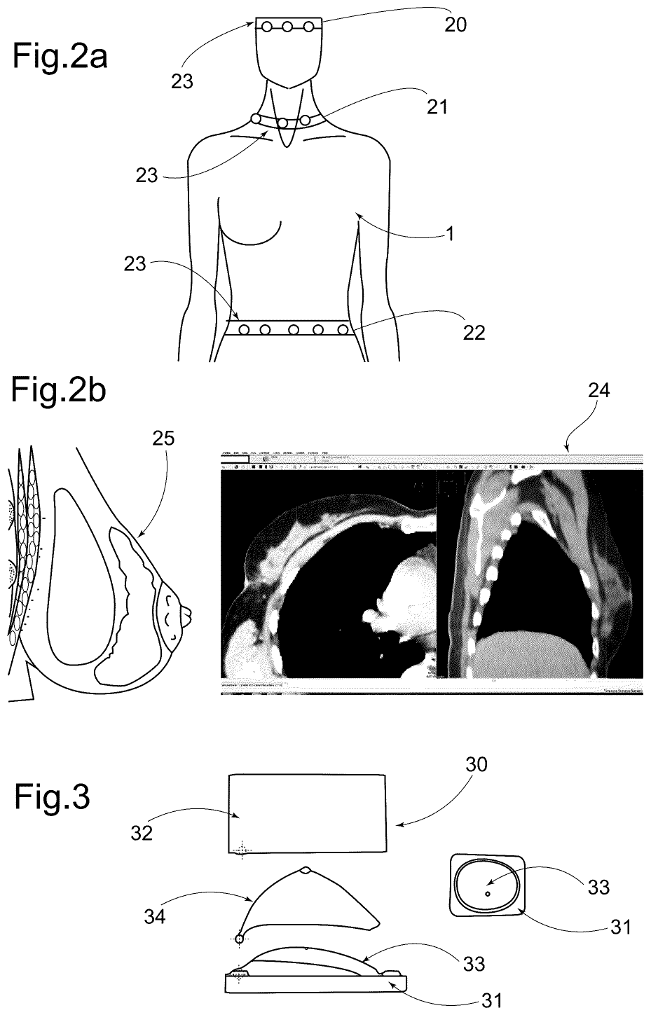

To enable the first and second digital volumetric scans 3 and 4 to be superimposed and processed by the three-dimensional CAD software mentioned above, the three-dimensional pre- and post-operative volumetric scans must provide positioning marks. One possible solution for ensuring this positioning of the two consecutive scans is illustrated in FIG. 2a. In this solution, the first and second digital volume scans 3 and 4 are taken with markers positioned on the upper part of the body of patient 1 on which the three-dimensional volume scans are taken. In this example of implementation of this part of the invention, these markers are placed on the patient before carrying out the first and second digital volume measurements 3 and 4. They take the form, for example, of a headband 20, a collar 21 and a belt 22. Each of these marking elements has static markings 23. For example, the headband and collar each have three static markers 23, and the belt has five. These static markers 23 enable the first and second digital volumetric representations 3 and 4 to be superimposed perfectly, and processing of the corresponding files by the three-dimensional CAD software makes it possible to obtain the digital volumetric representation 5, which reproduces with a very high level of accuracy the removed anatomical part that the anatomical prosthesis is to replace.

As illustrated in FIG. 2b, a CT scan 24 taken before the mastectomy is also taken into account in the method of the invention to obtain an even more faithful reproduction of the anatomical part to be replaced by the anatomical prosthesis. The CT scan 24 is performed, for example, with a CT scanner or by MRI (Magnetic Resonance Imaging). Preferably, this tomo-densitometric scan 24 will be processed by densitometric transmission software to obtain a digital tomographic scan 25. Thanks to this digital tomographic record 25, the distribution of tissue density in the breast to be removed is reproduced identically in the anatomical prosthesis intended to replace it after the mastectomy. Advantageously, the CT scan 24 is carried out during the first appointment with the patient with the locating elements 20 to 22 shown in FIG. 2a positioned on the part of the body of the patient 1.

In certain special cases, for example when it has not been possible to carry out the preliminary phase of preoperative three-dimensional recording illustrated in FIG. 1a, or in cases of bilateral mastectomy, it is possible to simulate the envelope of the volume formed by the patient's morphology before the mastectomy. In the case of a unilateral mastectomy, a three-dimensional volume file of the patient's anatomy before the mastectomy can be produced by symmetric transcription of the volume envelope of the part of the patient's anatomy not affected by the mastectomy. This three-dimensional file obtained by symmetry may be used instead of the first digital volume record 3. In the case of bilateral mastectomy, a three-dimensional volume file of the patient's anatomy before mastectomy may be produced on the basis of a set of pre-registered standard measurements corresponding to the volume envelope of the patient's morphology. In this situation, the three-dimensional file obtained by virtual reconstruction on the basis of standard measurements is used in place of the first digital volume measurement 3 after validation by the patient.

Alternatively, standard volume representations 5 can be superimposed on the patient's second digital volume record 4 in order to define the digital volume representation that best corresponds to the patient's anatomy, and after matching the corresponding digital files, prostheses appropriate to the patient's anatomy can be formalised by computer-aided 3D design without using the first digital volume record 3. In the case of the ablation of a single breast without it having been possible to carry out the first digital volumetric scan 3, the digital volumetric representation 5 of the anatomical prosthesis to be produced is carried out by 3D CAD on the basis of the unremoved breast. In this way, the method of the invention makes it possible to deal with all the situations and challenges of postoperative reconstruction using an external prosthesis, and can also be applied to situations in which the first digital volume scan cannot be carried out, for example in cases of accidental mutilation.

As indicated above, in the process described for producing anatomical prostheses, in this case breast prostheses, the various digital files resulting from the various scans carried out on the patient before and after the surgical procedure are transposed and formatted to enable additive manufacturing, also known as 3D printing, using for example an SLA, FDM or SLS process, of a mould or directly of the anatomical prosthesis, whether temporary, prototypical or definitive, as explained below.

FIG. 3 shows a mould 30 and its construction. In this first example of anatomical prosthesis manufacture, the digital file corresponding to the digital volume representation 5 is transposed and formatted so that it can be used by a 3D printer to produce, by additive manufacturing, a mould into which the flexible synthetic material making up the anatomical prosthesis, for example a biomedical silicone, will be injected. The mechanical properties that the mould 30 must have mean that rigid synthetic materials such as ABS, PLA, PP, nylon, etc. must be used. To achieve this, mould 30 can be obtained by one of three types of 3D printing, i.e. FDM, SLA or SLS.

The production of an anatomical prosthesis by injecting biomedical silicone into a mould 30 obtained by 3D printing from the digital volumetric representation 5, makes it possible to obtain an anatomical prosthesis that identically reproduces the removed anatomical part. The silicone injected or cast into the mould 30 will be coloured in the mass in order to faithfully reproduce the skin tone of the anatomical part removed on the basis of the digital colour measurement carried out on the patient.

The mould 30 can be made in two parts, a base 31 and an injection volume 32 to produce an anatomical silicone prosthesis poured or injected into the mould 30. In this case, the density of the anatomical prosthesis will be defined from the digital tomographic scan 25. The density of the anatomical prosthesis can be defined as the average density of the densities recorded in the digital tomographic record 25.

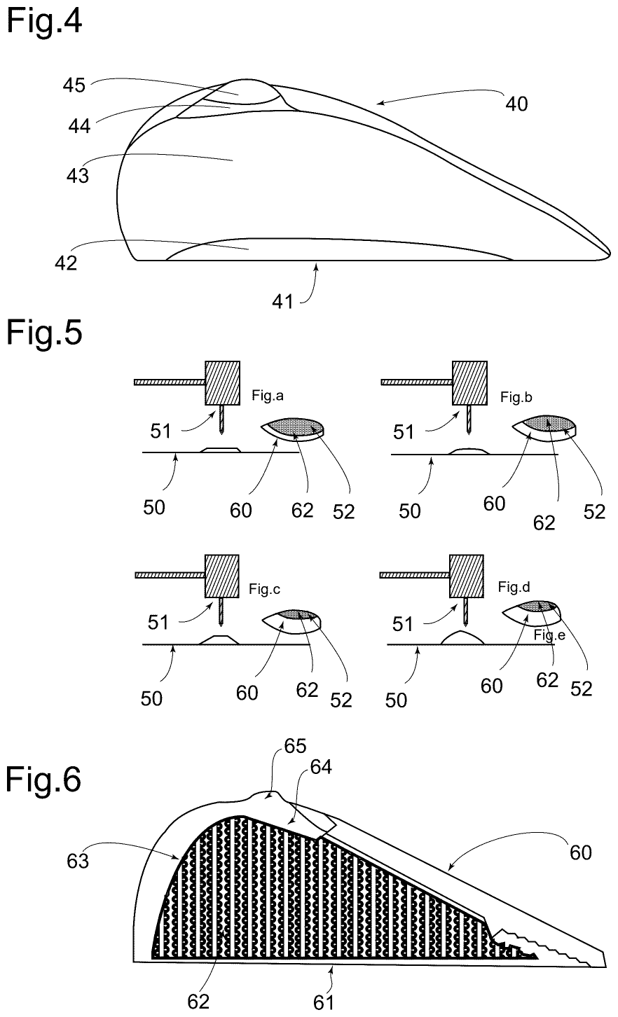

FIG. 3 shows a more than two-part mould 30 for producing an anatomical multi-density silicone prosthesis. As explained above, the mould 30 has a mould base 31 on which an impression of the scar 33 is formed as a protuberance which faithfully represents the topography of the area of the patient's body in which the scar 6 is located as digitised in the digital volume representation 5 from the second digital volume scan 4 carried out after the mastectomy on the part of the patient's body 1. Thus, as illustrated by FIG. 4, a multi-density moulded anatomical prosthesis 40 has a moulded base 41 intended to be brought into contact with the patient's skin and to closely match the topography of the area containing the scar 6 after the time required for complete post-operative healing and after the end of the radiotherapy or chemotherapy following the mastectomy.

The multi-density moulded anatomical prosthesis 40 comprises different zones in which silicones of different structures have been injected into the mould 30 according to the densities transposed from the digital tomographic scan 25. The multi-density moulded anatomical prosthesis 40 shown in FIG. 4 comprises a lower zone 42 in which the imprint of the scar 33 on the base of the mould 31 will have reproduced in negative the topography of the zone of the patient's body in which the scar 6 is located. The multi-density moulded anatomical prosthesis 40 also comprises an internal zone 43, an areolar zone 44 and a nipple zone 45. Each of these zones 41 to 45 of the multi-density moulded anatomical prosthesis 40 has a specific density determined from the density of the respective zone of the breast which has been removed from the sensitometric measurements recorded in the digital tomographic record 25.

To produce the multi-density moulded anatomical prosthesis 40, a mould 30 is used in which an inner mould part 34 can be positioned on the base of the mould 31. In this case, several inner mould parts 34 are necessary to obtain a multi-density moulded anatomical prosthesis 40. For example, to produce the multi-density moulded anatomical prosthesis 40 shown in FIG. 4, a first inner mould part 34 will be positioned first, leaving only the volume corresponding to the nipple zone 45 free in the volume of the mould 32, into which a silicone having the density of the nipple zone will be injected. In a second step, the first inner mould part 34 is replaced by a second inner mould part, the shape of which makes it possible to inject a silicone, the density of which corresponds to the density of the areolar zone 44, in order to produce the areolar zone 44. A third inner part of the mould is used to inject a silicone of the desired density to produce the inner zone 43. The lower zone 42 is created last without inserting an inner mould part 34.

Once the monodensity or pluridensity moulded anatomical prosthesis (40) has been produced, a finishing step can be carried out by applying a final covering agent enabling all the external details recorded in the centimetric photographic record to be reproduced. The covering agent used for this finishing step may be a medical silicone with different extrinsic pigmentations which is applied, for example, with an airbrush or a brush.

FIG. 5 illustrates various stages in the production of a printed anatomical prosthesis 60 by 3D printing, preferably by SLA or FDM type 3D printing as shown here. The anatomical prosthesis can be obtained directly by 3D printing by formatting the file of the digital vomic representation 5 so that a 3D printer produces an anatomical prosthesis identically reproducing the volume of the anatomical part removed. The biomedical silicone used for this purpose can be tinted using the digital colour map so that the anatomical prosthesis also reproduces the patient's skin tone.

Due to the complexity of the shape of the anatomical prosthesis, FDM or SLA 3D printing is particularly suitable for the manufacture of anatomical prostheses, in particular SLA 3D printing due to the fact that a printed base 61 may have to represent in negative the topography of the area of the patient's body in which the scar 6 is located as digitised in the digital volume representation 5.

3D printing makes it possible to produce both single-density and multi-density anatomical prostheses by varying the internal structure of the anatomical prosthesis according to the data from the digital tomographic scan 25. The internal structure of the prosthesis can take different forms. FIG. 5 shows an anatomical prosthesis being printed with a lattice structure 62. The printed base 61 of the anatomical prosthesis rests on a printing plate 50 of a 3D printer 51, for example of the FDM type, on which a printing base (not shown) is positioned which can represent the topography of the scar site 6 and on which the volume of the anatomical prosthesis 60 will be printed. The print head 52 supplies the filament which is melted at the locations where the material is to be deposited layer by layer on the print plate 50.

FIG. 6 shows a cross-section of a printed anatomical prosthesis 60. The internal structure has a lattice 62 produced by 3D printing, the structure of which makes it possible to reproduce the different density measurements recorded in the digital tomographic scan. A peripheral structure 63 reproduces the skin tone recorded in the digital colorimetric scan 25 as well as the peripheral density of the removed anatomical part. The same applies to an areolar area 64 and a nipple area 65. The printed base 61 reproduces in negative the topography of the patient's anatomical area including the scar 6 so that the printed base closely matches the shape of the patient's scar 6.

3D printing makes it possible to directly produce a printed anatomical prosthesis 60 reproducing identically all the external and internal details of the removed breast, including those recorded during the centimetric photographic scan. Optionally, the external details recorded in the centrimetric photographic scan can be produced during a finishing step, which can be carried out manually using, for example, an airbrush spraying biomedical silicones dyed to reproduce these external details, or a brush.

For example, when the patient can tolerate a so-called definitive anatomical prosthesis, such as the printed anatomical prosthesis 60 described above, this can be printed in a very realistic manner identical to the anatomical part whose shape has been digitised in the digital volume representation 5. The process of the invention then makes it possible to generate contour joints 66 enabling the prosthesis to be applied very closely to the patient's body so as to render its contours virtually invisible thanks to the customised colour and texture of the printed anatomical prosthesis 60. The process for obtaining an anatomical prosthesis by moulding described above also makes it possible to obtain a definitive prosthesis of this type.

The methods of implementation of the process described above enable the volume, skin tone and density of the breast to be reproduced by 3D printing. In fact, the internal masses recorded by body densitometry will be transposed by software to represent the data recorded inside the breast before its ablation or the contralateral breast when necessary.

Before producing the definitive anatomical prosthesis described above, it may be appropriate to produce a silicone or resin prototype in order to validate the volume of the anatomical prosthesis after visualising the volume representing it. The finishing stage with the covering agent, and the final adaptation on the patient in order to touch up and refine all the possible characteristics, in particular the contour joint 66, can thus be carried out during or after validation of the shape of the anatomical prosthesis. Visualisation and validation of a prototypical anatomical prosthesis are particularly important when the first digital volume scan 3 could not be carried out before the mastectomy.

Before being able to benefit from a definitive printed or moulded anatomical prosthesis, a temporary prosthesis is offered to patients in the healing phase and during post-operative radiotherapy or chemotherapy treatments during which patients cannot tolerate contact between the prosthesis and the skin.

Nevertheless, so that these patients can benefit rapidly after the mastectomy from an individual prosthesis corresponding to their anatomy, the methods for implementing the invention make it possible to produce provisional anatomical prostheses that are realistic and adapted to the patient's morphology, but that can be used in bras instead of being bonded by medical adhesive directly to the patient's skin. These temporary prostheses are adapted to be received in a specially designed textile pocket in the patient's underwear. This temporary solution allows patients to benefit from the technology described above and thus retain their own anatomy, compared with current standard solutions which do not reproduce the patient's anatomy identically.

During the healing phase or during radiotherapy and chemotherapy treatments, patients will have no problem tolerating these temporary anatomical prostheses until they are able to tolerate the definitive anatomical prosthesis, which is positioned close to the skin and can be bonded using a biomedical adhesive or a self-adhesive sheet to hold the prosthesis directly to the patient's body.

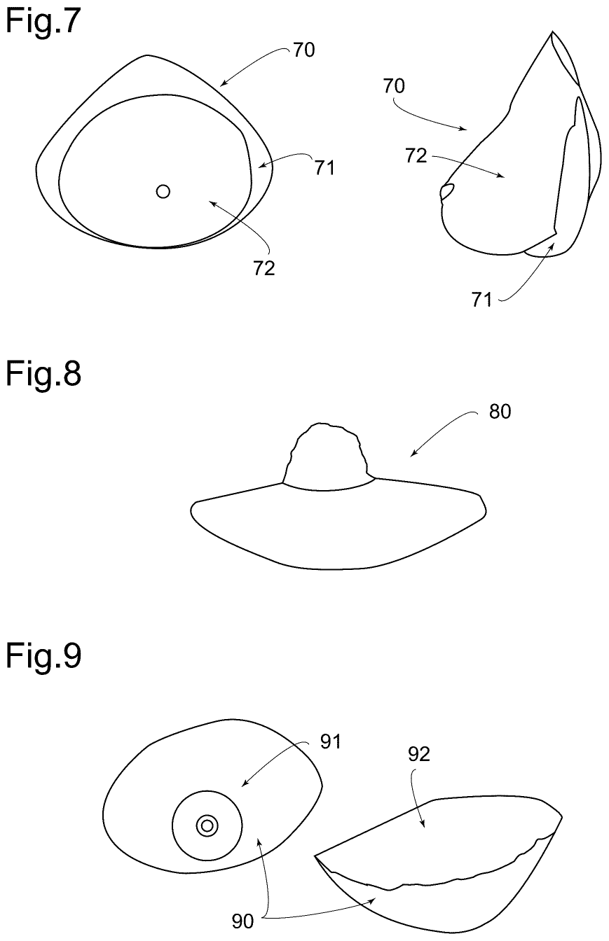

FIG. 7 shows a provisional anatomical prosthesis 70 produced by moulding or 3D printing as described above and illustrated in FIGS. 3 and 5. This provisional anatomical prosthesis 70 is intended to be integrated into an underwear pocket in order to be worn in particular during the months following the mastectomy. The provisional anatomical prosthesis 70 is generally delivered approximately two months after the mastectomy. It can therefore be used as a provisional model pending complete post-operative healing, or pending reconstruction using an internal prosthesis. The provisional anatomical prosthesis 70 can also be used as a prototype with a view to finalising the definitive anatomical prosthesis.

A template 71 is first selected, for example on the basis of the digital volume representation 5, from the virtual templates corresponding to the various existing breast sizes. Next, a volume of provisional prosthesis 72 formalised by the digital volume representation 5 is digitally merged with the pattern 71. The result of this digital file fusion is used as a basis for producing the provisional anatomical prosthesis 70 by moulding or directly by 3D printing.

The temporary anatomical prosthesis 70 is worn in a suitable bra. The volume of the provisional prosthesis 72 is not in direct contact with the patient's skin, as it is the pattern 71 which will face the skin inside the underwear. Thus the topography of the base of the volume of the temporary prosthesis 72 does not need to be adapted to the shape of the scar 6. The volume of the provisional prosthesis 72 is injected or printed from a biomedical silicone with a monodensity corresponding to the average density of the breast removed from each patient, or a pluridensity corresponding to the data from the digital tomographic scan 25.

Similarly, the skin tone of the provisional anatomical prosthesis 70 is represented by the shade of the silicone injected or poured into the mould 30, or used for 3D printing. This shade can be individual or selected from a choice of colours representing the usual skin colours on the basis of the digital colorimetric scan. A final covering agent can be applied to the volume of the provisional prosthesis 72 to finalise the external aspects of the provisional anatomical prosthesis 70.

Nowadays, advances in cancer diagnosis mean that total mastectomy is increasingly avoided, and only partial mastectomy or lumpectomy is performed, which generally involves ablation of the nipple and areola. In addition, advances in surgical techniques mean that reconstruction using an internal prosthesis is more common where the nipple is missing. As a result, the nipple and areola areas are increasingly in demand, as they are among the most important elements for the realism of all breast prostheses.

FIG. 8 shows a nipple and areola prosthesis 80, which is prescribed following a lumpectomy where the nipple and areola are removed, or in the case where they are missing following surgical reconstruction by internal prosthesis after a mastectomy. Thus, the process according to the invention makes it possible to obtain a conceptualised and formalised nipple and areola prosthesis 80 by moulding or 3D printing, as described above, so that it can be integrated into the patient's remaining anatomy to reproduce her pre-operation anatomy identically.

FIG. 9 shows a overlying breast prosthesis 90. Following a partial mastectomy, for example the ablation of half a breast, or in the case of a total mastectomy followed by reconstruction using an internal prosthesis, the overlying breast prosthesis 90 may be prescribed. The overlying breast prosthesis 90 is produced by moulding or 3D printing according to the invention process described above in order to reproduce identically the half of the breast removed. An external surface 91 of the overlying breast prosthesis 90 reproduces identically the nipple, areola and external details of the half of the breast removed using the digital tools described above. These tools also make it possible to create an inner surface 92 which will be in intimate contact with the remaining part of the breast and which will hide the scar 6. To achieve this, the inner surface 92 can reproduce in negative the topography of the area of the breast containing the scar 6.

The invention therefore makes it possible to produce anatomical prostheses, such as definitive or provisional breast prostheses, by reproducing them by direct 3D printing or by injection into a mould produced by 3D printing. In both cases, the envelope of the volume obtained by 3D printing is produced identically to the envelope of the volume of the anatomical part to be removed from a digital volume representation 5 of the volume of the removed anatomical part to be replaced by the anatomical prosthesis 2. This digital volume representation 5 is obtained by 3D CAD by superimposing a first digital volume scan 3 produced before the surgical procedure or a 3D simulation of the part of the patient's body before the surgical procedure, and a second digital volume scan 4 produced after the surgical procedure.

To further enhance the realism of the anatomical prosthesis 2, particularly when it is a definitive prosthesis, the distribution of densities within the anatomical prosthesis 2 or the internal structure of the anatomical prosthesis is reproduced from the digital tomographic scan 25. The same applies to the visual realism of the anatomical prosthesis 2, which can be improved by reproducing the patient's skin tone from the colourimetric scan and/or the centimetric photographic scan of the anatomical part to be removed. The various sets of digital data from the tomographic scan, the colorimetric scan and/or the photocentimetric scan can be used to complete the first digital volume scan 3 or the volume representation 5.

The development of the various methods of implementing the process for producing anatomical prostheses is intended to provide physiological and psychological repair for patients who have been amputated or mutilated, in particular patients who have undergone a mastectomy or lumpectomy following breast cancer.

As indicated in the preceding description, the various aspects of the invention can be used to produce prostheses other than breast prostheses without going beyond the scope of the invention. For example, the process of the invention makes it possible to produce an epithesis to replace a nose or an ear reproducing the density of the tissues of which it is composed, as well as the cartilaginous parts, identically or at least realistically if the mutilated part could not be scanned beforehand. According to another example, the invention process enables an amputated limb to be reproduced identically by modifying the moulded or injected materials, in particular their density and rigidity. In this way, the anatomical prosthesis will faithfully reproduce the volumes reproducing the muscles as well as those reproducing the bone structure of the amputated limb.

Naturally, the invention is described in the foregoing by way of example. It is understood that the person skilled in the art will be able to produce different variants of the invention and to implement it for other purposes.

1. Method of producing an anatomical prosthesis of flexible synthetic material by three-dimensional computer-aided design on the basis:
a first digital volume measurement (3) obtained by three-dimensional digitisation performed by three-dimensional surface scanning of an envelope of the volume of at least one part of the body of a patient (1) before ablation of an anatomical part to be replaced by said anatomical prosthesis (2), or by digital simulation of the envelope of the volume of at least one part of the body of a patient (1) before ablation of an anatomical part to be replaced by said anatomical prosthesis (2), and
a second digital volume record (4) obtained by three-dimensional digitisation performed by three-dimensional surface scanning of an envelope of the volume of the same at least one part of the patient's body (1) after ablation of the anatomical part to be replaced by the said anatomical prosthesis (2); and
in which a digital volumetric representation (5) of an envelope of the volume of the anatomical prosthesis (2) is obtained from the superposition of the first and second digital volumetric scans (3; 4) so as to reproduce identically an envelope of the anatomical part to be replaced by said anatomical prosthesis (2).

2. Method according to claim 1, characterised in that:
the external visual appearance of the anatomical prosthesis (2) reproduces identically the visual appearance of the anatomical part to be replaced by said anatomical prosthesis (2) on the basis of a digital colorimetric scan and/or a centimetric photographic scan of the anatomical part to be replaced by said anatomical prosthesis (2) before its ablation, preferably said digital colorimetric scan centimetric photographic scan complements said digital volumetric representation (5); and/or
the density of the volume of the said anatomical prosthesis (2) is defined on the basis of a digital tomographic scan (25) of at least part of the patient's body (1) taken before ablation of the anatomical part to be replaced by the said anatomical prosthesis (2), preferably the said digital colourimetric scan and/or the said centimetric photographic scan supplement the said digital volume representation (5).

3. Process according to one of claim 1 or 2 in which:
a base of the said anatomical prosthesis oriented towards the patient's skin is fused to a pattern (70) whose size corresponds to that of the said base of the anatomical prosthesis; or
a base (41; 61) of said anatomical prosthesis (2) intended to be in contact with the patient's skin has a topography obtained from said second digital volumetric scan (5) so as to correspond identically to the profile of an area of the patient's body from which the anatomical part to be replaced by said anatomical prosthesis (2) has been removed.

4. Method according to one of claims 1 to 3, characterised in that a mould (30) is made on the basis of said digital volume representation (5), into which the flexible synthetic material is injected to form said volume of said anatomical prosthesis (2).

5. Method according to claim 4, characterised in that the said mould (30) is produced by additive manufacturing from the digital volume representation (5).

6. Method according to one of claim 4 or 5, characterised in that the flexible synthetic material injected into the mould (30) has:
a skin tone corresponding to the digital colorimetric scan; and/or
a density corresponding to the average density of the digital tomographic image (25), or a density distribution derived from the digital tomographic image (25).

7. Method according to any one of the preceding claims, characterised in that a finishing step makes it possible to reproduce, after the moulding of the anatomical prosthesis (2), the pigmentation of the various areas of the volume of the said anatomical prosthesis (2) identical to the anatomical part to be replaced by the anatomical prosthesis (2) on the basis of the digital colorimetric scan and/or the centimetric photographic scan.

8. Method according to one of claim 1 or 2, characterised in that the anatomical prosthesis (2) is produced by additive manufacturing on the basis of:
of said digital volume representation (5) so that the shape of the volume of said anatomical prosthesis (2) is identical to the shape of the anatomical part to be replaced by said anatomical prosthesis (2);
said digital colorimetric scan and/or said centimetric photographic scan so that the visual appearance of said anatomical prosthesis (2) is identical to the visual appearance of the removed anatomical part; and/or
of the digital tomographic scan (25) so that the density distribution in the said anatomical prosthesis (2) is identical to the density distribution in the anatomical part to be replaced by the said digital prosthesis (2).

9. Method according to one of the preceding claims, characterised in that the flexible synthetic material used is a biomedical silicone dyed in the mass in order to reproduce the colours at the tone and saturation level required by the digital colorimetric scan.

10. Anatomical prosthesis produced by moulding or by additive manufacturing, characterised in that it is produced on the basis of a digital volume representation (5) of an anatomical prosthesis volume obtained by superposition:
a first digital volume measurement (3) obtained by three-dimensional digitisation performed by three-dimensional surface scanning of an envelope of the volume of at least one part of the body of a patient (1) before ablation of an anatomical part to be replaced by said anatomical prosthesis (2), or by digital simulation of the envelope of said at least one part of the body of a patient (1) before ablation of an anatomical part to be replaced by said anatomical prosthesis (2), and
a second digital volume record (4) obtained by three-dimensional digitisation performed by three-dimensional surface scanning of an envelope of the volume of the same said at least one part of the patient's body (1) after ablation of the anatomical part to be replaced by the said anatomical prosthesis (2).

11. Anatomical prosthesis according to claim 10, characterized in that:
the external visual appearance of the anatomical prosthesis (2) reproduces identically the visual appearance of the anatomical part to be replaced by said anatomical prosthesis (2) on the basis of a digital colorimetric scan and/or a centimetric photographic scan of the anatomical part to be replaced by said anatomical prosthesis (2); and/or
the volume density of said anatomical prosthesis (2) is defined on the basis of a digital tomographic scan (25) of at least part of the patient's body (1) taken before ablation of the anatomical part to be replaced by said anatomical prosthesis (2).

12. A method of modelling an anatomical prosthesis (2) by computer-aided three-dimensional design on the basis of a digital modelling file comprising first digital volume representation data (5) of an envelope of the anatomical prosthesis obtained by superposition:
a first digital volume measurement (3) obtained by three-dimensional digitisation performed by three-dimensional surface scanning of an envelope of the volume of at least one part of the body of a patient (1) before ablation of an anatomical part to be replaced by said anatomical prosthesis (2), or by digital simulation of the envelope of the volume of at least one part of the body of a patient (1) before ablation of an anatomical part to be replaced by said anatomical prosthesis (2), and
a second digital volume record (4) obtained by three-dimensional digitisation performed by three-dimensional surface scanning of an envelope of the volume of the same said at least one part of the patient's body (1) after ablation of the anatomical part to be replaced by the said anatomical prosthesis (2).

13. A method of modelling an anatomical prosthesis as claimed in claim 12, characterised in that the digital modelling file further comprises:
second data corresponding to a digital colorimetric scan and/or a centrimetric photographic scan of the anatomical part to be replaced by said anatomical prosthesis (2) prior to its ablation; and/or
third data corresponding to a digital tomographic scan (25) of at least part of the patient's body (1) prior to ablation of the anatomical part to be replaced by said anatomical prosthesis (2).
